(19) United States
(12) Patent Application Publication
(10) Pub. No.: US 2022/0117719 A1
(43) Pub. Date: Apr. 21, 2022

(54) PULSATILE VASCULAR STENT GRAFT

(71) Applicant: Leonhardt Ventures LLC, Mission Viejo, CA (US)

(72) Inventor: Howard J. Leonhardt, Mission Viejo, CA (US)

(21) Appl. No.: 17/500,795

(22) Filed: Oct. 13, 2021

Related U.S. Application Data
(60) Provisional application No. 63/094,529, filed on Oct. 21, 2020.

(51) Int. Cl. A61F 2/07; A61N 1/40; A61N 1/36

(52) U.S. Cl. CPC A61F 2/07; A61F 2250/0043; A61N 1/36002; A61N 1/40

(57) ABSTRACT

Described is an intravascular device including a stent structure and at least one annular band. The stent structure may be sized for insertion into a blood vessel and configured to expand to contact the wall of a blood vessel after insertion therein. The annular band is configured to change in diameter in response to an applied electrical field. A method of maintaining and accelerating pulsatile blood flow includes positioning an annular band within a blood vessel, and causing the annular band to expand and contract in response to an electrical field.

### CROSS-REFERENCE TO RELATED APPLICATIONS

This application claims the benefit under 35 U.S.C. § 119(e) of U.S. Provisional Patent Application Ser. No. 63/094,529, filed Oct. 22, 2020, the disclosure of which is incorporated herein in their entirety by this reference.

### TECHNICAL FIELD

The application, in various embodiments, relates generally to intravascular devices and related methods and systems. More particularly, the disclosure relates to intravascular devices that include an annular band configured to change shape in response to an applied electrical or other appropriate field, and related methods and systems.

### BACKGROUND

Some internal circulatory assist devices have impellers (see, e.g., PCT International Patent Publication WO2019183247A1 to Leonhardt (Sep. 26, 2019), the contents of which are incorporated by reference).

Sometimes, impellers may damage red blood cells and often reduce pulsaltility of both blood flow and blood vessel wall movement. Additionally, internal circulatory assist devices often have blood clots form within them thus blocking flow.

External vessel systems may cause damage to blood vessels, are inconsistent in performance and have to be placed surgically with invasive procedures. Some circulatory assist devices migrate out of position, or require suture sewing, hooks, and/or barbs to be held in place. Additionally many circulatory assist devices or are too rigid to maintain vessel wall pulsaltility. Accordingly, such external devices may damage the blood vessels and are inconsistent in performance and must be placed surgically.

Many internal devices cause damage to blood cells and have a relatively high risk of blood clotting and most often eliminate vessel wall or blood flow pulsaltility.

### BRIEF SUMMARY

In some embodiments of the disclosure, an intravascular device includes a stent structure and an annular band. The stent structure is preferably sized for insertion into a blood vessel and configured to expand to contact the wall of a blood vessel after insertion therein so as to be maintained in position. The annular band may be configured to change in diameter in response to an applied (e.g., electrical or magnetic) field.

In certain embodiments, described are intravascular devices including an annular band configured to change shape in response to an applied field, methods for maintaining and accelerating pulsatile blood flow, and related systems.

In further embodiments of the disclosure, a method of maintaining and accelerating pulsatile blood flow may include positioning an annular band within a blood vessel, and causing the annular band to expand and contract in response to an, e.g., electrical field.

### BRIEF DESCRIPTION OF THE DRAWINGS

While this disclosure concludes with claims particularly pointing out and distinctly claiming specific embodiments, various features and advantages of embodiments within the scope of this disclosure may be more readily ascertained from the following description when read in conjunction with the accompanying drawings, in which:

FIG. 1 is a side elevation view of an intravascular device according to an embodiment of the disclosure;

FIG. 2 is a partial cross-sectional view of a catheter being used to insert the intravascular device of FIG. 1 into a blood vessel;

FIG. 3 is a partial cross-sectional view of the intravascular device of FIG. 1 positioned within the blood vessel in a partially actuated mode; and

FIG. 4 is a partial cross-sectional view of the intravascular device of FIG. 1 positioned within the blood vessel in a fully actuated mode.

### DETAILED DESCRIPTION

In the following detailed description, reference is made to the accompanying drawings, which form a part hereof, and in which are shown, by way of illustration, specific examples of embodiments in which the disclosure may be practiced. These embodiments are described in sufficient detail to enable a person of ordinary skill in the art to practice the disclosure. However, other embodiments enabled herein may be utilized, and structural, material, and process changes may be made without departing from the scope of the disclosure.

The illustrations presented herein are not necessarily meant to be actual views of any particular method, system, device, or structure, but are merely idealized representations that are employed to describe the embodiments of the disclosure. In some instances similar structures or components in the various drawings may retain the same or similar numbering for the convenience of the reader; however, the similarity in numbering does not necessarily mean that the structures or components are identical in size, composition, configuration, or any other property.

The following description may include examples to help enable one of ordinary skill in the art to practice the disclosed embodiments. The use of the terms “exemplary,” “by example,” and “for example,” means that the related description is explanatory, and though the scope of the disclosure is intended to encompass the examples and legal equivalents, the use of such terms is not intended to limit the scope of an embodiment or this disclosure to the specified components, steps, features, functions, or the like.

It will be readily understood that the components of the embodiments as generally described herein and illustrated in the drawings could be arranged and designed in a wide variety of different configurations. Thus, the following description of various embodiments is not intended to limit the scope of the disclosure, but is merely representative of various embodiments. While the various aspects of the embodiments may be presented in the drawings, the drawings are not necessarily drawn to scale unless specifically indicated.

Furthermore, specific implementations shown and described are only examples and should not be construed as the only way to implement the disclosure unless specified otherwise herein. For the most part, details concerning timing considerations and the like have been omitted where such details are not necessary to obtain a complete understanding of the disclosure and are within the abilities of persons of ordinary skill in the relevant art.

Any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. In addition, unless stated otherwise, a set of elements may comprise one or more elements.

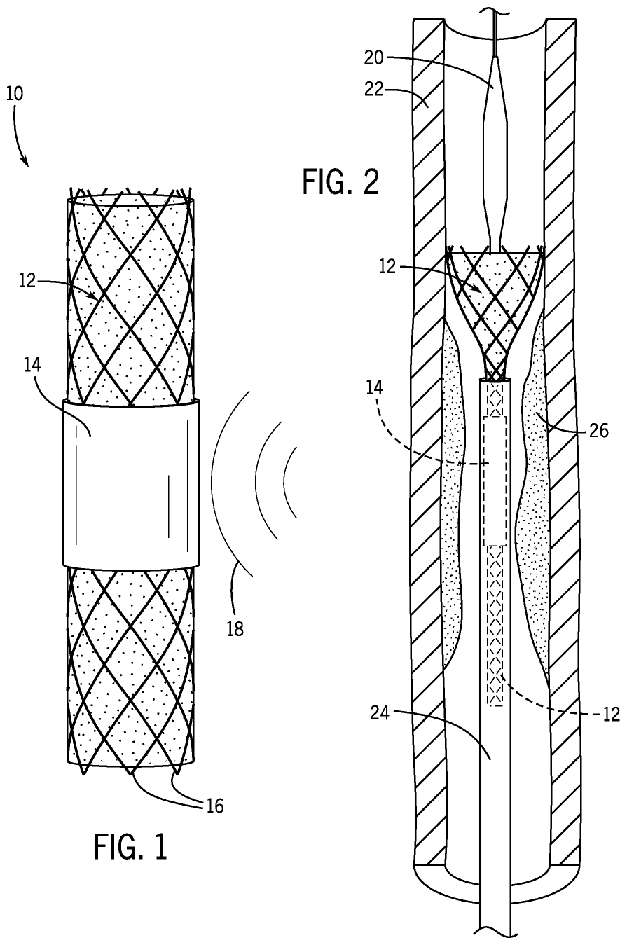

FIG. 1 shows an intravascular device 10 according to an embodiment of the disclosure, generally in an inactive mode. The depicted intravascular device includes a stent structure 12 and at least one annular band 14 that is configured to be electronically activated. In some embodiments, the annular band 14 may be an electro-activated polymer, such as a ferroelectric polymer or a dielectric polymer. Accordingly, an electrical (or, e.g., magnetic) field may be applied to the annular band 14 and the annular band 14 will change shape (e.g., expand and/or contract) in response to the applied electric field (e.g., a voltage).

In certain embodiments, rather than an electrical field, a magnetic field, or ultrasonic or light energy field is utilized to activate the band(s) and/or stent.

The stent structure 12 is preferably sized for insertion into a blood vessel and configured to expand to contact the wall of a blood vessel after insertion therein. The stent structure 12 may include nitinol wires with alternating bends to form a zigzag or other shape extending circumferentially around the stent structure 12 and a graft material, such as expanded polytetrafluoroethylene (ePTFE), which covers the wires and may serve as an artificial blood vessel wall.

In certain embodiments, the stent itself pulsates; not just the annular band. See, e.g., Palma et al. “Pulsatile stent graft: a new alternative in chronic ventricular assistance” Brazilian Journal of Cardiovascular Surgery (2013), 28(2): 217-223, the contents of which are incorporated herein by this reference. In Palma et al., pulsatile stents composed of nickel-titanium were built and positioned to engage latex tubes simulating the aorta. Different electric currents were applied to units connected in series in order to cause structure contraction and displacement of a liquid column. There were two sequence tests: first composed of two metallic cages and the second composed of five cages. At first sequence tests was applied a voltage of 16.3 volts and a current of 5 amperes. In the second, voltage of 15 volts and current of 07 amperes. In the first sequence was obtained the pulsatile effect of stent, with contraction of the tube and displacement of the water column sufficient to validate the pulsating effect of the endoprosthesis. The two structures ejected a volume of 2.6 ml per cycle, with a range of 29 mm in height of the column of water equivalent to 8% shrinkage during the pulse. In the second sequence, it reached a variation of 7.4 mL per cycle. The results obtained confirm the stent pulsatile contractility activated by electrical current.

In certain embodiments, the stent graft is a GORE® stent graft with the center stent replaced with a piezo electric band. In certain embodiments, the stent graft further includes a metallic mesh covered by ePTFE (expanded polytetrafluoroethylene (PTFE) available from Gore). See, e.g., Rosset et al. “Mechanical properties of electroactive polymer microactuators with ion-implanted electrodes” Electroactive Polymer Actuators and Devices (EAPAD) 2007, Proc. of SPIE Vol. 6524, 652410, (2007) doi: 10.1117/12.714944, the contents of which are incorporated herein by this reference.

WO 2006123317A2 to Dubois et al. (Mar. 1, 2007), the contents of which are incorporated herein by this reference, discloses a dielectric electroactive polymer comprising an elastomer layer arranged between two compliant elastomer electrodes wherein at least one of the compliant elastomer electrodes is obtained by ion implantation on the elastomer layer. The dielectric electroactive polymer may be used in an actuator, sensor, or in a power source. Also disclosed is a process for manufacturing a dielectric electroactive polymer.

In some embodiments, the annular band 14 may comprise a dielectric polymer. An outer conductive layer may be positioned on or proximate to the outer surface of the annular band 14, and an inner conductive layer may be positioned on or proximate to the inner surface of the annular band 14. The dielectric polymer of the annular band may be positioned between the inner conductive layer and the outer conductive layer. Accordingly, when a field (e.g., an electrical field such as a voltage) is applied to the inner conductive layer and the outer conductive layer of the annular band 14 the electric field may cause the inner conductive layer and the outer conductive layer of the annular band 14 to be attracted toward each other or repulsed away from each other.

For example, an electric field may cause the inner conductive layer and the outer conductive layer of the annular band 14 to be attracted toward each other and the attraction of the inner conductive layer and the outer conductive layer of the annular band 14 may cause the dielectric polymer positioned in between to be compressed and thinned. The compression and thinning of the dielectric polymer of the annular band 14 may cause the diameter of the annular band to increase. When the electric field is removed or altered, the inner conductive layer and the outer conductive layer of the annular band 14 may no longer be attracted toward each other, and the elasticity of the dielectric polymer may cause the annular band 14 to return to its original shape.

For another example, an electric field may cause the inner conductive layer and the outer conductive layer of the annular band 14 to be repelled away from each other and the repulsion of the inner conductive layer and the outer conductive layer of the annular band 14 may cause the dielectric polymer positioned in between to be expanded and thickened. The expansion and thickening of the dielectric polymer of the annular band 14 may cause the diameter of the annular band to decrease. When the electric field is removed or altered, the inner conductive layer and the outer conductive layer of the annular band 14 may no longer be repelled away from each other, and the elasticity of the dielectric polymer may cause the annular band 14 to return to its original shape.

In further embodiments, the annular band 14 may comprise a ferroelectric polymer, such as polyvinylidene fluoride (PVDF). Accordingly, when an electric field (e.g., a voltage) is applied to the annular band 14 the electric field may change the organization of the molecular dipole of the ferroelectric polymer causing a change in shape of the annular band 14. For example, the reorganization of the molecular dipoles of the ferroelectric polymer by the applied electric field may cause the annular band 14 to contract or expand. When the electric field is removed or altered, the original molecular dipole organization of the ferroelectric polymer may be at least partially restored and the annular band 14 may return to its original shape.

As shown in FIG. 2, the intravascular device 10 may be delivered to a location within a blood vessel 22 with a catheter 24 and a guide wire 20. For example, the intravascular device 10 may be delivered to a location in the blood vessel 22 with a buildup of plaque 26.

Prior to insertion of the catheter into a patient, the intravascular device 10 may be compressed and inserted into the catheter 24. The alternating bends in the stent wire may allow the radial compression of the stent 12 like a spring, and the flexible polymer materials of the graft and the annular band 14 may allow sufficient deformation for positioning into the catheter 24. The guide wire 20 may also be positioned within the catheter 24 and extend through the intravascular device 10.

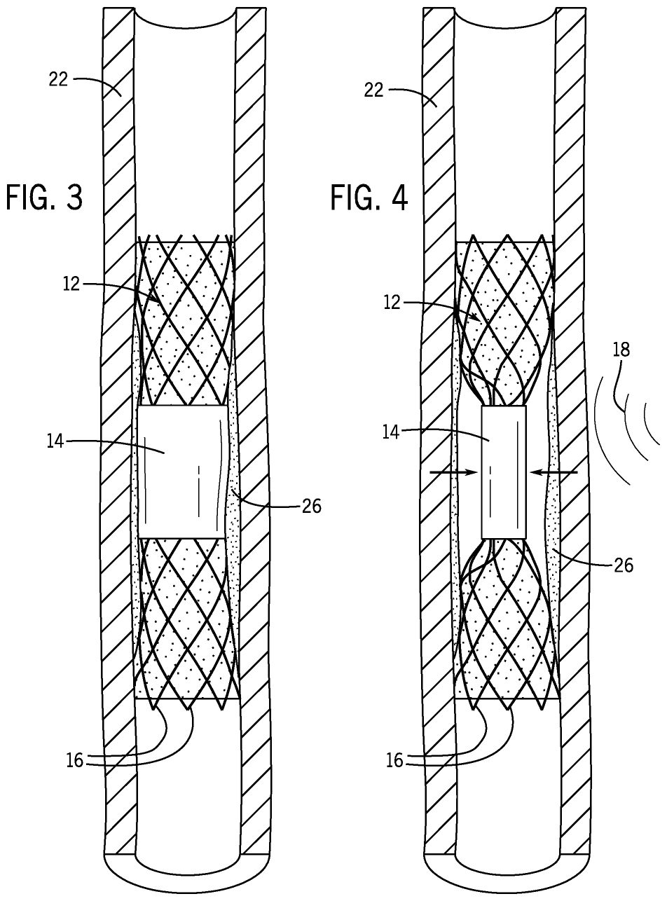

After insertion into a patient, the tip of the catheter may be guided to the desired location within the blood vessel 22 with the assistance of the guide wire 20. The intravascular device 10 may then be deployed out of the tip of the catheter 24. As the stent 12 portion of the intravascular device 10 exits the catheter 24, the wires at the distal end may rebound like a spring and expand to cause a first end of the stent 12 to contact the wall of the blood vessel 22. Additionally, the stent 12 may expand within the annular band 14 as the annular band 14 is deployed from the catheter 24 and expand the annular band within the blood vessel 22. As the distal end of the stent 12 exits the catheter 24, the wires at the distal end may rebound like a spring and expand to cause a second end of the stent 12 to contact the wall of the blood vessel 22, as shown in FIG. 3. After the intravascular device 10 has been deployed to the desired location within the blood vessel 22, the catheter 24 and the guide wire 20 (see FIG. 2) may be withdrawn from the patient.

After the intravascular device 10 has been deployed to the desired location within the blood vessel 22, the annular band 14 may be actuated to alternate between a first shape, such as shown in FIG. 3, and a second shape, such as shown in FIG. 4, in response to an applied electric field to maintain and/or accelerate pulsatile blood flow. The stent 12 may provide a spring radial force at the ends of the intravascular device 10 with sufficient force to secure the intravascular device 10 firmly in the blood vessel 22 without preventing vessel wall pulsaltility. Additionally, any spring force applied by the stent 12 under the annular band 14 may be not be so strong as to prevent the shape change of the annular band 14 when the electric field is applied.

In some embodiments, the annular band 14 of the intravascular device 10 may be actuated by an electric field created by a device positioned outside of the patient's body. For example, electric current may be directed through a conductive coil located outside of the patient's body at a location proximal to the intravascular device 10 to generate an electric field that may be directed at the annular band 14 of the intravascular device 10 to activate the annular band 14 of the intravascular device 10. For example, an external belt or other securing device worn by a patient may include an electric field generator configured to deliver wireless electro-magnetic energy 18 on demand in pulses to cause the annular band 14 to pulsate at a chosen frequency, which can be timed with the electrocardiogram (ECG) of the patient with delay built in. It may be understood, however, that any chosen frequency may be selected. Accordingly, the intravascular device 10 may placed in a desired blood vessel and is used to augment blood flow providing circulatory assist support.

In some embodiments, the intravascular device 10 may be used in the aorta just above the renal arteries to help heart failure patients with excess body fluid to remove that fluid by accelerating pulsatile flow into the kidneys. In some embodiments, the intravascular device 10 may be used in legs with low blood flow to avoid limb amputation. In further embodiments, the intravascular device 10 may be used in hemodialysis patients to avoid blood clot formations in arteriovenous grafts and fistulas as well as central venous lines.

Intravascular devices according to embodiments of the disclosure may be used in any application in any field where it is desired to move fluid particularly if pulsaltility of flow is desired. For example, intravascular devices according to embodiments of the disclosure may be utilized for: an aorta circulatory assist pump, a heart wrap heart assist, leg and foot circulatory flow improvement, improving blood flow into kidneys via renal artery placement, improving blood flow to the eyes, improving blood flow to the brain, improving blood vessel compliance to reduce high blood pressure, improving strength and breathing of airway tubes, reducing blood clots during hemodialysis, and improving drug delivery including cancer therapies.

Embodiments of the disclosure may offer improvements over other circulatory assist devices that have impellers which damage red blood cells and often reduce pulsaltility of both blood flow and blood vessel wall movement. Such devices often have blood clots form within them thus blocking flow. External vessel systems can cause damage to blood vessels, are inconsistent in performance and have to be placed via invasive surgery. Other devices migrate out of position or require suture sewing or hooks/barbs to be held in place or are too rigid to maintain vessel wall pulsaltility. Embodiments of the disclosure may avoid such problems.

Accordingly, embodiments of the disclosure may accelerate pulsatile blood flow with minimization of hemolysis and blood clot formation and without eliminating pulsatile vessel wall movement.

While the disclosure is susceptible to various modifications and alternative forms, specific embodiments have been shown by way of example in the drawings and have been described in detail herein. However, the disclosure is not intended to be limited to the particular forms disclosed. Rather, the disclosure is to cover all modifications, equivalents, and alternatives falling within the scope of the disclosure as defined by the following appended claims and their equivalents.

### REFERENCES (THE CONTENTS OF EACH OF WHICH ARE INCORPORATED HEREIN BY THIS REFERENCE)

Khan, Sadeque Reza et al. “Wireless Power Transfer Techniques for Implantable Medical Devices: A Review.” Sensors (Basel, Switzerland) vol. 20, 12, 3487 (19 Jun. 2020), doi:10.3390/s20123487.
Palma et al. “Pulsatile stent graft: a new alternative in chronic ventricular assistance,” Revista Brasileira de Cirurgia Cardiovascular (2013), 28(2):217; dx.doi.org/10.5935/1678-9741.20130031.
Rosset et al. “Mechanical properties of electroactive polymer microactuators with ion-implanted electrodes” Electroactive Polymer Actuators and Devices (EAPAD) 2007, Proc. of SPIE Vol. 6524, 652410, (2007) doi: 10.1117/12.714944.
U.S. Pat. No. 8,585,571 to Bachman (Nov. 19, 2013) for “Portable controller with integral power source for mechanical circulation support systems.”
U.S. Pat. No. 9,002,469 to D'Ambrosio (Apr. 7, 2015) for “Transcutaneous energy transfer system with multiple secondary coils.”
U.S. Pat. No. 9,271,825 to Arkusz et al. (Mar. 1, 2016) for “Pulsating Stent Graft”.
U.S. Pat. No. 9,308,303 to Badstilbner et al. (Apr. 12, 2016) for “Transcutaneous power transmission and communication for implanted heart assist and other devices.”
U.S. Pat. No. 9,642,958 to Zilbershlag et al. (May 9, 2017) for “Coplanar Wireless Energy Transfer”.
U.S. Pat. No. 9,855,376 to Bluvshtein et al. (Jan. 2, 2018) for “Power Scaling”.
U.S. Pat. No. 10,149,933 to Bluvshtein et al. (Dec. 11, 2018) for “Coil parameters and control”.
WO 2006123317A2 to Dubois et al. (Mar. 1, 2007).

What is claimed is:

1. An intravascular device comprising:
a stent structure sized for insertion into a blood vessel and configured to expand to contact the wall of a blood vessel after insertion therein; and
at least one annular band configured to change in diameter in response to an applied field.

2. The intravascular device of claim 1, wherein the annular band is configured to change in diameter in response to an applied electrical field.

3. The intravascular device of claim 1, wherein the annular band is comprised of an electro-activated polymer.

4. The intravascular device of claim 1, wherein the annular band is comprised of a dielectric polymer.

5. The intravascular device of claim 1, wherein the annular band is comprised of a ferroelectric polymer.

6. A method of maintaining and accelerating pulsatile blood flow in a subject, the method comprising:
positioning an annular band within a blood vessel of the subject; and
causing at least one annular band to expand and contract in response to an applied field.

7. The method according to claim 6, wherein the applied field is an electrical field.

8. The method according to claim 5, wherein positioning the annular band within a blood vessel comprises positioning the annular band within the blood vessel with a catheter.

9. The method according to claim 7, further comprising directing the electrical field wirelessly to the annular band from a source located outside of a subject's body.

10. The method according to claim 9, further comprising delivering the electric field on demand in pulses to cause the at least one annular band to pulsate at a chosen frequency.

11. The method according to claim 10, further comprising timing the pulses with an electrocardiogram of the patient.

12. The method according to claim 6, wherein the blood vessel is an aorta or renal artery.

13. The method according to claim 12, further comprising improving blood flow into the kidneys.

14. The method according to claim 6, wherein positioning the annular band within a blood vessel comprises positioning the annular band within a blood vessel of the legs.

15. The method according to claim 14, further comprising improving leg and foot circulatory flow.

16. The method according to claim 6, further comprising improving blood flow to the eyes or brain.

17. The method according to claim 6, further comprising improving blood vessel compliance to reduce high blood pressure.

18. The method according to claim 6, further comprising reducing blood clots during hemodialysis.

19. The method according to claim 6, further comprising improving drug delivery.

20. The method according to claim 19, wherein improving drug delivery comprises improving the delivery of cancer therapies.
